(19) United States
(12) Patent Application Publication
(10) Pub. No.: US 2026/0057513 A1
(43) Pub. Date: Feb. 26, 2026

(54) SYSTEMS AND METHODS FOR MEDICAL IMAGE PROCESSING

(71) Applicant: SHANGHAI UNITED IMAGING HEALTHCARE CO., LTD., Shanghai (CN)

(72) Inventors: Chen XI, Shanghai (CN); Yang LV, Shanghai (CN); Yun DONG, Shanghai (CN)

(21) Appl. No.: 19/308,390

(22) Filed: Aug. 25, 2025

(30) Foreign Application Priority Data
Aug. 23, 2024 (CN) 202411174291.6

(51) Int. Cl. G06T 7/00; G16H 30/40

(52) U.S. Cl. CPC G06T 7/0012; G16H 30/40; G06T 2207/20081

(57) ABSTRACT

A system and a method for medical image processing are provided. The method includes: obtaining a first medical image; obtaining a trained image perception restoration model; the trained image perception restoration model includes an image quality perception sub-model and an image restoration sub-model; and inputting the first medical image into the trained image perception restoration model to obtain a second medical image. The image quality perception sub-model is configured to determine either or both of a first quality evaluation value of the first medical image and a second quality evaluation value of the second medical image, the image restoration sub-model is configured to determine the second medical image, and the quality of the second medical image is higher than that of the first medical image.

### CROSS-REFERENCE TO RELATED APPLICATION

This application claims priority to Chinese Patent Application No. 202411174291.6, filed on Aug. 23, 2024, the entire contents of which are hereby incorporated by reference.

### TECHNICAL FIELD

The present disclosure relates to field of medical imaging technology, and in particular, to methods, systems, devices, and storage medium for medical image processing.

### BACKGROUND

Quality control of medical images is generally complex. In practical scenarios, a variety of medical equipment is usually involved, such as computed tomography (CT) equipment, magnetic resonance imaging (MRI) equipment, positron emission tomography (PET) equipment, digital subtraction angiography (DSA) equipment, and the like. The image quality varies among different medical devices or different models of the same type of medical device. How to unify image quality or monitor the status of equipment through the perception of image quality is a complex issue.

Therefore, it is desirable to provide methods, systems, devices, and storage medium for addressing the problem of inconsistent image quality caused by different devices or different user perception habits existing in the prior art.

### SUMMARY

An aspect of the present disclosure provides a method for medical image processing, implemented on a machine including one or more processors and one or more storage devices, the method including: obtaining a first medical image; obtaining a trained image perception restoration model; the trained image perception restoration model includes an image quality perception sub-model and an image restoration sub-model; and inputting the first medical image into the trained image perception restoration model to obtain a second medical image. The image quality perception sub-model is configured to determine either or both of a first quality evaluation value of the first medical image and a second quality evaluation value of the second medical image, the image restoration sub-model is configured to determine the second medical image, and the quality of the second medical image is higher than that of the first medical image.

In some embodiments, the first quality evaluation value is configured to evaluate the quality of the first medical image, the second quality evaluation value is configured to evaluate the quality of the second medical image, and the second quality evaluation value is higher than the first quality evaluation value.

In some embodiments, the image quality perception sub-model includes a first image quality perception sub-model and a second image quality perception sub-model, the first quality evaluation value of the first medical image is determined by the first image quality perception sub-model, and the second quality evaluation value of the second medical image is determined by the second image quality perception sub-model.

In some embodiments, network parameters of the first image quality perception sub-model and the second image quality perception sub-model in the trained image perception restoration model are the same.

In some embodiments, the image quality perception sub-model is configured to determine the second quality evaluation value of the second medical image includes: determining the second medical image by the image restoration sub-model, and inputting the second medical image into the image quality perception sub-model to obtain the second quality evaluation value of the second medical image.

In some embodiments, the method further includes: constructing a training dataset, the training dataset including first label data, the first label data including a first sample image, a second sample image, a first sample evaluation value of the first sample image, and a second sample evaluation value of the second sample image; the quality of the second sample image being higher than that of the first sample image; and training image perception restoration model based on the training dataset to obtain the trained image perception restoration model.

In some embodiments, the training dataset further includes second label data; the second label data includes a third sample image and a third sample evaluation value of the third sample image; and training image perception restoration model according to the training dataset to obtain the trained image perception restoration model further includes training image perception restoration model according to the first label data to obtain a first image perception restoration model; and training the image quality perception sub-model of the first image perception restoration model according to the second label data to obtain the trained image perception restoration model.

In some embodiments, the training dataset further includes third label data; the third label data includes a fourth sample image and a fifth sample image; the quality of the fifth sample image is higher than that of the fourth sample image; and the training image perception restoration model according to the training dataset to obtain the trained image perception restoration model further includes: training image perception restoration model according to the first label data to obtain a first image perception restoration model; and training the image restoration sub-model of the first image perception restoration model according to the third label data to obtain the trained image perception restoration model.

In some embodiments, the method further includes: constructing a training dataset, the training dataset including fourth label data and fifth label data; the fourth label data including a sixth sample image and a sixth sample evaluation value of the sixth sample image; the fifth label data including a seventh sample image and an eighth sample image; the quality of the eighth sample image being higher than that of the seventh sample image; and training image perception restoration model based on the training dataset to obtain the trained image perception restoration model.

In some embodiments, the training image perception restoration model according to the training dataset to obtain the trained image perception restoration model further includes: training the image quality perception sub-model and the image restoration sub-model of the image perception restoration model to obtain a second image perception restoration model; the image quality perception sub-model being trained according to the fourth label data and the image restoration sub-model being trained according to the fifth label data; and obtaining the trained image perception restoration model based on the second image perception restoration model.

In some embodiments, the training dataset further includes sixth label data; the sixth label data includes a ninth sample image, a tenth sample image, a ninth sample evaluation value of the ninth sample image, and a tenth sample evaluation value of the tenth sample image; the quality of the tenth sample image being higher than that of the ninth sample image; and the obtaining the trained image perception restoration model based on the second image perception restoration model further includes: training the second image perception restoration model according to the sixth label data to obtain the trained image perception restoration model.

Another aspect of the present disclosure provides a system for medical image processing, including: at least one storage devices including a set of instructions; and at least one processor in communication with the at least one storage devices. When executing the set of instructions, the at least one processor is configured to cause the system to perform the following operations: obtaining a first medical image; obtaining a trained image quality perception sub-model and a trained image restoration sub-model; inputting the first medical image into the trained image restoration sub-model to obtain a second medical image; the quality of the second medical image being higher than that of the first medical image; and either or both of inputting the first medical image into the trained image quality perception sub-model to obtain a first quality evaluation value of the first medical image and inputting the second medical image into the trained image quality perception sub-model to obtain a second quality evaluation value of the second medical image.

In some embodiments, the trained image quality perception sub-model includes a third image quality perception sub-model and a fourth image quality perception sub-model, the first quality evaluation value of the first medical image is determined by the third image quality perception sub-model, and the second quality evaluation value of the second medical image is determined by the fourth image quality perception sub-model.

In some embodiments, network parameters of the third image quality perception sub-model and the fourth image quality perception sub-model in the trained image quality perception sub-model are the same.

In some embodiments, the at least one processor is further configured to cause the system to perform the following operations: obtaining a trained image perception restoration model based on the trained image quality perception sub-model and the trained image restoration sub-model.

Another aspect of the present disclosure provides a method for medical image processing, including: obtaining at least one first medical image; obtaining a trained image perception restoration model; the trained image perception restoration model includes an image quality perception sub-model and an image restoration sub-model; and inputting the at least one first medical image into the trained image perception restoration model to obtain at least one second medical image. The quality of each of the second medical images meets a preset quality requirement.

In some embodiments, the at least one first medical image of a plurality of first medical images with different qualities; and the method further includes: inputting the plurality of first medical images with different qualities into the trained image perception restoration model to obtain a plurality of second medical images, and the plurality of second medical images meet the same quality requirement.

In some embodiments, the image quality perception sub-model includes a first image quality perception sub-model and a second image quality perception sub-model, a first quality evaluation value of the first medical image is determined by the first image quality perception sub-model, and a second quality evaluation value of the second medical image is determined by the second image quality perception sub-model.

In some embodiments, network parameters of the first image quality perception sub-model and the second image quality perception sub-model in the trained image perception restoration model are the same.

In some embodiments, the method further includes: inputting the at least one second medical image into the image quality perception sub-model to obtain the second quality evaluation value of the second medical image.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further illustrated in terms of exemplary embodiments. These exemplary embodiments are described in detail with reference to according to the drawings. These embodiments are non-limiting exemplary embodiments, in which like reference numerals represent similar structures.

FIG. 1 is a hardware structure block diagram of a terminal for implementing the medical image processing method according to an embodiment of the present disclosure.

FIG. 2 is a flowchart of a medical image processing method according to an embodiment of the present disclosure.

FIG. 3 is a schematic diagram of a model application according to an embodiment of the present disclosure.

FIG. 4 is a schematic diagram of another model application according to an embodiment of the present disclosure.

FIG. 5 is a schematic diagram of yet another model application according to an embodiment of the present disclosure.

FIG. 6 is a flowchart of another medical image processing method according to an embodiment of the present disclosure.

FIG. 7 is a flowchart of yet another medical image processing method according to an embodiment of the present disclosure.

FIG. 8 is a flowchart of still another medical image processing method according to an embodiment of the present disclosure.

FIG. 9 is a flowchart of a further medical image processing method according to an embodiment of the present disclosure.

FIG. 10 is a structural schematic diagram of an image perception restoration model according to an embodiment of the present disclosure.

FIG. 11 is an application schematic diagram according to an embodiment of the present disclosure.

FIG. 12 is a structural block diagram of a medical image processing apparatus according to an embodiment of the present disclosure.

### DETAILED DESCRIPTION OF THE EMBODIMENT

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but is to be accorded the widest scope consistent with the claims.

The terminology used herein is for the purpose of describing particular example embodiments only and is not intended to be limiting. As used herein, the singular forms “a”, “an”, and “the” may be intended to include the plural forms as well, unless the context clearly indicates otherwise. It will be further understood that the terms “comprise”, “comprises”, and/or “comprising”, “include”, “includes”, and/or “including”, when used in this specification, specify the presence of stated features, integers, steps, operations, elements, and/or components, but do not preclude the presence or addition of one or more other features, integers, steps, operations, elements, components, and/or groups thereof.

It will be understood that the terms “system”, “engine”, “unit”, “module”, and/or “block” used herein are one method to distinguish different components, elements, parts, sections or assemblies of different levels in ascending order. However, the terms may be displaced by another expression if they achieve the same purpose.

Generally, the word “module”, “unit”, or “block”, as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on”, “connected to”, or “coupled to”, another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The terms “pixel” and “voxel” in the present disclosure are used interchangeably to refer to an element of an image. These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

In general, medical image quality control is relatively complex. In practical scenarios, it typically involves a variety of medical devices, such as computed tomography (CT) devices, magnetic resonance imaging (MRI) devices, positron emission tomography (PET) devices, digital subtraction angiography (DSA) devices, and the like. There are variations in image quality among different medical devices or among products of different models of the same type of medical device. How to standardize image quality or monitor device status through the perception of image quality is a complex issue.

Different users (e.g., physicians, technicians, etc.) may have different habits in perceiving image quality even when evaluating images acquired by the same device under the same acquisition conditions. This leads to inconsistencies in the judgment of image quality. If the quality of output images is maintained at a nearly consistent level, it will facilitate users' operations (e.g., diagnosis, teaching, etc.).

In addition, in the prior art, high-quality images are obtained by extending the scanning time of the subject, increasing the dose of drugs injected into the subject's body, and/or enhancing the time-of-flight (TOF) sensitivity of the imaging system used to scan the subject. However, these methods may increase the subject's radiation dose, make the images more susceptible to motion artifacts, and cause increased discomfort to the subject due to prolonged scanning time. Machine learning models (e.g., neural network models) are widely used for image optimization in medical imaging. For example, deep neural networks can be used for image denoising and/or enhancement, thereby improving the efficiency and accuracy of disease diagnosis and/or treatment.

Regarding the problems existing in the prior art, such as image quality inconsistencies caused by different devices or different users' perception habits, and increased radiation dose to the subject during the acquisition of high-quality images, no effective solution has been proposed so far.

Compared with the prior art, a medical image processing method, system, apparatus, and storage medium provided in the embodiments of the present disclosure operate as follows: obtaining a first medical image; obtaining a trained image perception restoration model; the trained image perception restoration model including an image quality perception sub-model and an image restoration sub-model; and inputting the first medical image into the trained image perception restoration model to obtain a second medical image. The image quality perception sub-model is configured to determine either or both of a first quality evaluation value of the first medical image and a second quality evaluation value of the second medical image, the image restoration sub-model is configured to determine the second medical image, and the quality of the second medical image is higher than that of the first medical image.

In this way, a medical image with optimized quality is obtained, and the medical image is evaluated using a unified quality evaluation standard. This addresses the problems existing in the prior art, such as inconsistent image quality caused by different devices or different users' perception habits, and increased radiation dose to the subject during the acquisition of high-quality images, thereby realizing the unified evaluation of medical image quality.

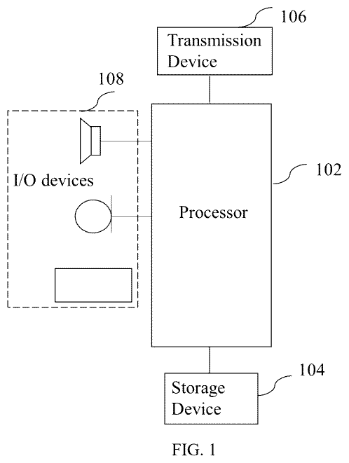

The method embodiments provided in the present disclosure may be implemented on terminals, computers, or similar computing devices. For example, when executed on a terminal, FIG. 1 illustrates a hardware architecture block diagram of a terminal configured to perform the medical image processing method according to an embodiment of the present disclosure. As shown in FIG. 1, the terminal includes one or more processors 102 (only one is depicted in the figure) and a memory 104 for data storage. The processor 102 may include, but is not limited to, processing devices such as a microcontroller unit (MCU) or a field-programmable gate array (FPGA). The terminal further incorporates a transmission device 106 for communication purposes and an input/output device 108. Those skilled in the art will appreciate that the structure shown in FIG. 1 is illustrative and does not limit the terminal's architecture—for instance, the terminal may include additional or fewer components than depicted, or feature a different configuration.

The memory 104 is configured to store computer programs, including software programs and modules of application software (e.g., the computer program corresponding to the medical image processing method of the present disclosure). The processor 102 executes various functional applications and data processing tasks (i.e., implements the claimed method) by running these computer programs stored in the memory 104. The memory 104 may include high-speed random-access memory (RAM) and non-volatile memory, such as one or more magnetic storage devices, flash memory, or other non-volatile solid-state storage media. In some implementations, the memory 104 may further include remotely located memories connected to the terminal via a network. Examples of such networks include, but are not limited to, the Internet, intranets, local area networks (LANs), mobile communication networks, and combinations thereof.

The transmission device 106 facilitates data reception and transmission over a network, which may include wireless networks provided by the terminal's communication service provider. In one example, the transmission device 106 includes a network interface controller (NIC) that connects to other network devices via a base station for Internet communication. In another example, the transmission device 106 may be a radio frequency (RF) module enabling wireless Internet connectivity.

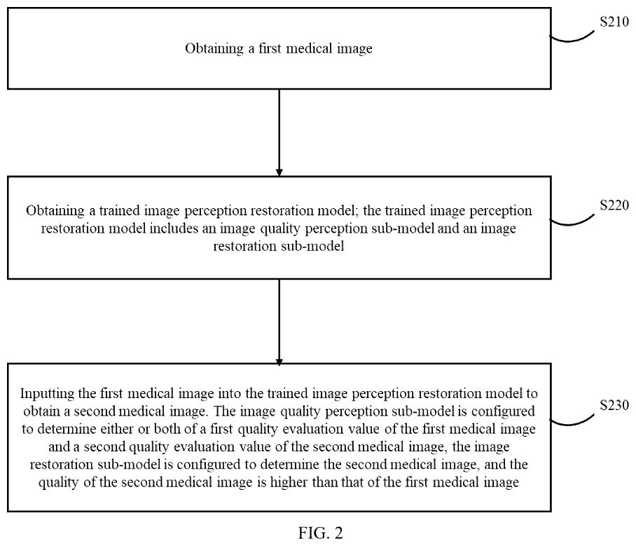

FIG. 2 illustrates a flowchart of a medical image processing method according to an embodiment of the present disclosure. As shown in FIG. 2, the process 200 includes the following step 210 and step 230.

Step 210, obtaining a first medical image.

In this step, the first medical image is a medical image requiring quality optimization and evaluation, which may exhibit quality issues such as noise or blur. The first medical image can be acquired from any modality of medical equipment. For example, if the medical device is a positron emission tomography (PET) device, the first medical image may be an image captured by a short-axis PET device or a long-axis PET device. The specific modality is not limited herein.

Step 220, obtaining a trained image perception restoration model; the trained image perception restoration model includes an image quality perception sub-model and an image restoration sub-model.

This step involves retrieving a trained image perception restoration model, which includes a first image quality perception sub-model and an image restoration sub-model. The trained image perception restoration model is trained using a large volume of medical image data to perform two primary functions: image quality evaluation of the input first medical image and/or the output second medical image; and image quality restoration of the first medical image to generate a second medical image with optimized quality.

The image quality perception sub-model evaluates the quality of the first medical image and/or the second medical image. By analyzing input image features through specific algorithms and model architectures, it outputs quality metrics reflecting image sharpness, noise level, contrast, and other quality attributes. These metrics provide quantitative assessments for medical professionals to validate image acceptability.

The image restoration sub-model enhances the quality of the first medical image through feature extraction, processing, and reconstruction. By mitigating issues like noise and blur, it improves critical quality indicators such as clarity and contrast, thereby generating the second medical image with optimized quality.

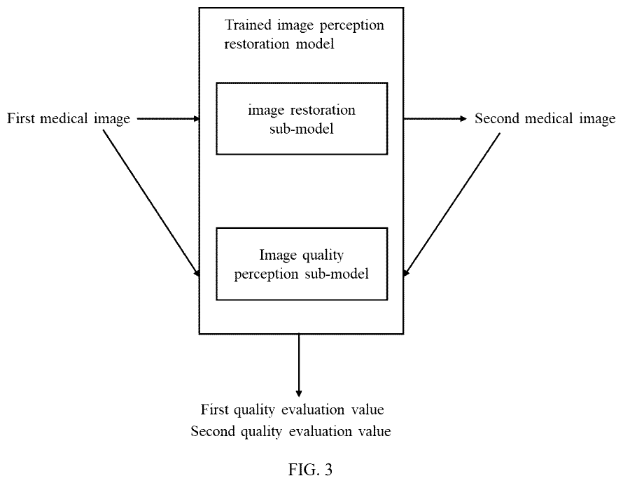

FIG. 3 is a schematic diagram of a model application according to an embodiment of the present disclosure. The trained image perception restoration model includes an image quality perception sub-model and an image restoration sub-model. During the training of the image perception restoration model, the image perception restoration model can be trained as a whole; alternatively, the image quality perception sub-model and the image restoration sub-model can be trained separately; it is also possible to first train the image perception restoration model as a whole, then fix the network parameters of either the image quality perception sub-model or the image restoration sub-model and fine-tune the network parameters of the other, so as to realize the training of the image perception restoration model; it is also possible to first train either the image quality perception sub-model or the image restoration sub-model in the image perception restoration model, fix its network parameters, then train the other, and finally fine-tune the network parameters of the image perception restoration model as a whole, so as to realize the training of the image perception restoration model. The specific training method is not limited herein.

The first medical image and the second medical image mentioned herein can be images acquired by medical equipment. The medical equipment herein can be computed tomography (CT) equipment, magnetic resonance imaging (MRI) equipment, positron emission tomography (PET) equipment, digital subtraction angiography (DSA) equipment, etc., which are not specifically limited herein. The quality of medical images acquired by different medical equipment or different models of the same medical equipment is different. For medical images acquired by different medical equipment or different models of the same medical equipment, the medical image processing method in the embodiment of the present disclosure needs to be adopted, and the image perception restoration model is configured to automatically realize image restoration and perform image quality evaluation, so as to unify the quality of medical images output by different medical equipment or different models of the same medical equipment, and unify the corresponding evaluation standards for image quality. The image perception restoration model herein can be various types of artificial intelligence models. Exemplarily, the image perception restoration model can include, but is not limited to, neural network models, generative adversarial network models, deep reinforcement learning models, fully supervised machine learning models, semi-supervised machine learning models, etc., or combinations thereof.

Step 230, inputting the first medical image into the trained image perception restoration model to obtain a second medical image. The image quality perception sub-model is configured to determine either or both of a first quality evaluation value of the first medical image and a second quality evaluation value of the second medical image, the image restoration sub-model is configured to determine the second medical image, and the quality of the second medical image is higher than that of the first medical image.

In this step, after obtaining the first medical image, the first medical image is input into the trained image perception restoration model to obtain a second medical image with optimized image quality. The image restoration sub-model is configured to optimize the image quality of the input first medical image, and the quality of the image optimized by the image restoration sub-model is higher than that of the image before optimization, that is, the quality of the second medical image is higher than that of the first medical image. In addition, the image quality perception sub-model is configured to evaluate the quality of the input first medical image and/or second medical image, so as to obtain a first quality evaluation value of the first medical image and/or a second quality evaluation value of the second medical image.

It should be noted that in some embodiments, the output result of the trained image perception restoration model may only include the second medical image with optimized image quality; in some embodiments, the output result of the trained image perception restoration model may include the second medical image with optimized image quality, the first quality evaluation value of the first medical image, and the second quality evaluation value of the second medical image; in some embodiments, the output result of the trained image perception restoration model may include the second medical image with optimized image quality and the first quality evaluation value of the first medical image; in some embodiments, the output result of the trained image perception restoration model may include the second medical image with optimized image quality and the second quality evaluation value of the second medical image.

In particular, during the training of the image perception restoration model, the image perception restoration model can first be trained as a whole based on the first label data (the first sample image, the second sample image, the evaluation value of the first sample image, and the evaluation value of the second sample image), and then the network parameters of either the image quality perception sub-model or the image restoration sub-model can be fixed, and the network parameters of the other can be fine-tuned. For example, fix the network parameters of the image restoration sub-model, and fine-tune the network parameters of the image quality perception sub-model based on the second label data (the first sample image and the evaluation value of the first sample image), so as to further improve the training accuracy of the image perception restoration model. The first sample image includes low-quality PET images, and the second sample image is a correspondingly optimized high-quality PET image. Based on this, the trained image perception restoration model will have the ability of image quality restoration (improvement of image quality). The image quality herein can refer to the resolution, clarity, etc. of the image. The fact that the quality of the second medical image is higher than that of the first medical image can mean that the clarity of the second medical image is higher than that of the first medical image, or that the resolution of the second medical image is higher than that of the first medical image.

As further shown in FIG. 3, the first medical image may be input into the image quality perception sub-model to obtain a quality evaluation value of the first medical image; alternatively, the second medical image with optimized image quality may be input into the image quality perception sub-model to obtain a quality evaluation value of the second medical image; in addition, both the first medical image and the second medical image may be input into the image quality perception sub-model to obtain quality evaluation values of the first medical image and the second medical image.

There is no sequential distinction between using the image quality perception sub-model to determine either or both of the first quality evaluation value of the first medical image and the second quality evaluation value of the second medical image, and using the image restoration sub-model to determine the second medical image. For example, in some embodiments, after obtaining the trained image perception restoration model, the first medical image may first be input into the image quality perception sub-model to obtain a quality evaluation value of the first medical image; then the first medical image is input into the image restoration sub-model to obtain the second medical image; finally, the second medical image is input into the image quality perception sub-model to obtain a quality evaluation value of the second medical image. In addition, the input of the image perception restoration model is the first medical image, and the output of the image perception restoration model may include the first quality evaluation value of the first medical image, the second medical image, and the second quality evaluation value of the second medical image; alternatively, the output may include the second medical image and the second quality evaluation value of the second medical image. Relevant descriptions have been provided earlier, and details will not be repeated here. Subsequently, the output of the image perception restoration model is transmitted to subsequent processing units or modules, facilitating users to perform data analysis and processing.

In this embodiment, by obtaining the trained image perception restoration model (which includes an image quality perception sub-model and an image restoration sub-model), the first medical image is input into the trained image perception restoration model to obtain the second medical image. Among them, the image quality perception sub-model is configured to determine either or both of the first quality evaluation value of the first medical image and the second quality evaluation value of the second medical image, the image restoration sub-model is configured to determine the second medical image, and the quality of the second medical image is higher than that of the first medical image. In this way, a medical image with optimized quality is obtained, and a unified quality evaluation standard is configured to evaluate the medical image with optimized quality. This solves the problems in the prior art, such as inconsistent image quality caused by different devices or different user perception habits, and increased radiation dose to the subject during the process of obtaining high-quality images, thereby realizing medical image optimization and quality evaluation.

In some embodiments, the first quality evaluation value is configured to evaluate the quality of the first medical image, the second quality evaluation value is configured to evaluate the quality of the second medical image, and the second quality evaluation value is higher than the first quality evaluation value.

The first quality evaluation value or the second quality evaluation value herein is configured to identify the image quality of the first medical image or the second medical image. The quality evaluation value may be determined by user habits and can be a specific numerical value-through a preset correspondence between numerical values and image quality, the image quality of the first medical image or the second medical image is identified. Alternatively, the quality evaluation value may be an image grade-through a preset correspondence between image grades and image quality, the image quality of the first medical image or the second medical image is identified. The image quality herein may refer to the resolution, clarity, etc., of the image. As a non-limiting example, the quality evaluation value is a specific score, which may include a 5-point scale, a 100-point scale, or other scoring scales. Taking the 5-point scale as an example, a lower score indicates poorer image quality. The image quality perception sub-model outputs a specific score on the 5-point scale to identify the image quality of the medical image. For instance, if the quality evaluation value corresponding to the first medical image is 1, it indicates that the first medical image is a low-quality image; if the quality evaluation value corresponding to the second medical image is 4 or 5, it indicates that the second medical image is a high-quality image and the image restoration meets expectations. The training dataset includes image quality scoring parts comprehensively provided by doctors, technicians, and researchers (e.g., subjective scores for image noise and image contrast). That is, the training dataset includes data of various scores attributed to the same scanned subject. Based on this, the trained image perception restoration model can automatically evaluate different image qualities and optimize image quality. The first medical images acquired or generated by different medical devices or different models of the same medical device are restored by the image perception restoration model to obtain second medical images with optimized quality. Moreover, the image perception restoration model is configured to evaluate either or both of the quality of the first medical images and the second medical images, obtaining quality evaluation values of the first medical images and/or the second medical images in accordance with a unified quality evaluation standard.

In some embodiments, the image quality perception sub-model includes a first image quality perception sub-model and a second image quality perception sub-model. The first quality evaluation value of the first medical image is determined by the first image quality perception sub-model, and the second quality evaluation value of the second medical image is determined by the second image quality perception sub-model.

In some embodiments, the network parameters of the first image quality perception sub-model and the second image quality perception sub-model in the trained image perception restoration model are the same.

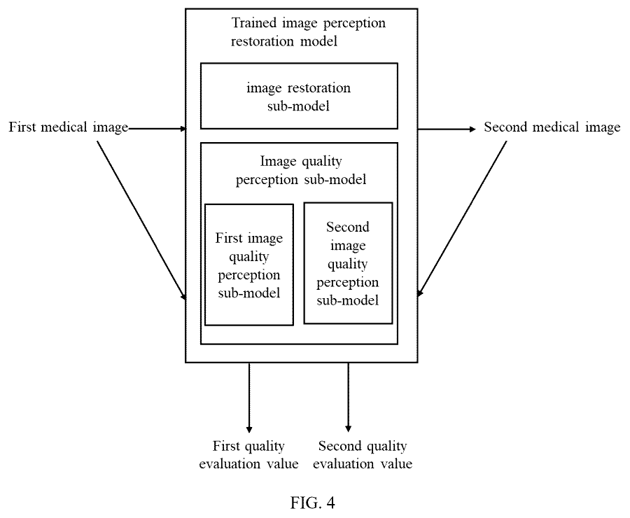

FIG. 4 is a schematic diagram of another model application according to an embodiment of the present disclosure. The image quality perception sub-model may include different image quality perception sub-models, such as a first image quality perception sub-model and a second image quality perception sub-model. In this case, the first quality evaluation value of the first medical image and the second quality evaluation value of the second medical image may be determined by different image quality perception sub-models respectively. The network parameters of the first image quality perception sub-model and the second image quality perception sub-model in the trained image perception restoration model may be the same. This structural design can enhance the model's ability to learn image features and improve the accuracy of image quality assessment. Additionally, in other embodiments, the first image quality perception sub-model and the second image quality perception sub-model may have different network parameters. In still other embodiments, the first image quality perception sub-model and the second image quality perception sub-model may adopt the same or different network structures.

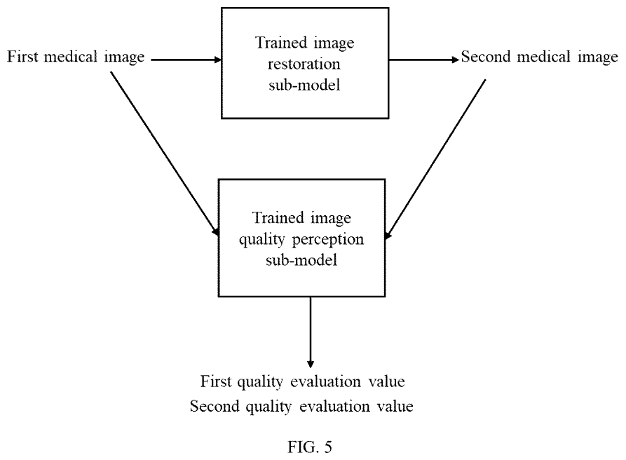

FIG. 5 is a schematic diagram of another model application according to an embodiment of the present disclosure. Beyond the above embodiments, in other embodiments, the image quality perception sub-model and the image restoration sub-model may also be used as independent models without being integrated into the image perception restoration model. For example, the image quality perception sub-model and the image restoration sub-model may be trained separately to obtain a trained image quality perception sub-model (for image quality evaluation) and a trained image restoration sub-model (for image quality optimization). For instance, the first medical image is input into the trained image restoration sub-model to obtain a second medical image with optimized quality; the first medical image and/or the second medical image is input into the trained image quality perception sub-model to obtain the first quality evaluation value of the first medical image and/or the second quality evaluation value of the second medical image. In some embodiments, determining the second quality evaluation value of the second medical image using the image quality perception sub-model includes: after determining the second medical image based on the image restoration sub-model, inputting the second medical image into the image quality perception sub-model to obtain the second quality evaluation value of the second medical image.

When the image quality perception sub-model includes only one model (i.e., a single image quality perception sub-model is required to determine both the first quality evaluation value of the first medical image and the second quality evaluation value of the second medical image), after the first medical image is input into the trained image perception restoration model, the first quality evaluation value of the first medical image may first be determined based on the image quality perception sub-model; at the same time, the second medical image with optimized quality is determined based on the image restoration sub-model; subsequently, the second medical image is input into the image quality perception sub-model to obtain the second quality evaluation value of the second medical image. In addition, the second medical image with optimized quality may first be determined based on the image restoration sub-model; then both the first medical image and the second medical image are input into the image quality perception sub-model to obtain the first quality evaluation value of the first medical image and the second quality evaluation value of the second medical image. Alternatively, only the second medical image may be input into the image quality perception sub-model to obtain the second quality evaluation value of the second medical image, without evaluating the quality of the first medical image.

In some of these embodiments, inputting the first medical image into the image quality perception sub-model to obtain the quality evaluation value of the first medical image includes: performing first feature extraction on the first medical image to obtain a first feature map; performing first feature processing on the first feature map to obtain a second feature map; and determining the quality evaluation value of the first medical image based on the second feature map.

In some of these embodiments, the first feature processing includes an adaptive pooling operation.

In this embodiment, first feature extraction is performed on the first medical image to obtain a first feature map. The convolutional layer in the image quality perception sub-model performs a convolution operation on the first medical image to extract image features, resulting in the first feature map. The convolution operation can capture both local and global features of the image, providing a basis for subsequent quality assessment.

First feature processing is performed on the first feature map to obtain a second feature map. For example, an adaptive pooling operation is performed: the adaptive pooling layer automatically adjusts the size and position of the pooling window according to the size of the input feature map and the preset output size, and performs a pooling operation on the first feature map to obtain the second feature map. The adaptive pooling operation can extract representative features from input feature maps of different sizes, enabling the model to have better adaptability to images of different sizes.

The quality evaluation value of the first medical image is determined based on the second feature map. The second feature map is further processed and analyzed through a fully connected layer or the like, and the feature map is mapped to a scalar value, which serves as the quality evaluation value of the first medical image. The weight parameters of the fully connected layer are adjusted through an optimization algorithm during the training process, so that the output quality evaluation value is as consistent as possible with the actual image quality.

In some of these embodiments, inputting the first medical image into the image restoration sub-model to obtain the second medical image includes: performing second feature extraction on the first medical image to obtain a third feature map; performing second feature processing on the first feature map to obtain a fourth feature map; concatenating the third feature map and the fourth feature map to obtain a fifth feature map; and obtaining the second medical image based on the fifth feature map.

In some of these embodiments, the second feature processing includes an upsampling operation.

In this embodiment, second feature extraction is performed on the first medical image. The convolutional layer in the image restoration sub-model performs a convolution operation on the first medical image to extract image features, resulting in the third feature map. The difference between the first feature extraction and the second feature extraction lies in the difference in the feature size of the feature maps. In this embodiment, the size of the third feature map is larger than that of the first feature map. Parameters such as the size, number, and stride of the convolution kernels are determined based on model design and experimental optimization. Different convolution kernels can extract features of different aspects of the image, such as edges and textures.

Second feature processing is performed on the first feature map. For example, an up-sampling operation is performed: the first feature map is up-sampled through a convolutional layer to increase the resolution of the feature map, resulting in the fourth feature map. The up-sampling operation can make the size of the feature map closer to that of the original image, facilitating subsequent image reconstruction.

The third feature map and the fourth feature map are concatenated to obtain a fifth feature map. The third feature map and the fourth feature map are merged in the channel dimension through channel concatenation, fusing feature information of different levels.

The second medical image is obtained based on the fifth feature map. The fifth feature map is further processed and reconstructed through a series of convolutional layers, activation function layers, etc., and finally the second medical image with higher quality is output. This image has significant improvements in clarity, noise level, and other aspects.

It should be noted that the feature maps in the embodiments of the present disclosure refer to the feature maps of the medical image network during forward propagation. The first feature map, second feature map, third feature map, fourth feature map, and fifth feature map represent feature maps at different layer positions in the network.

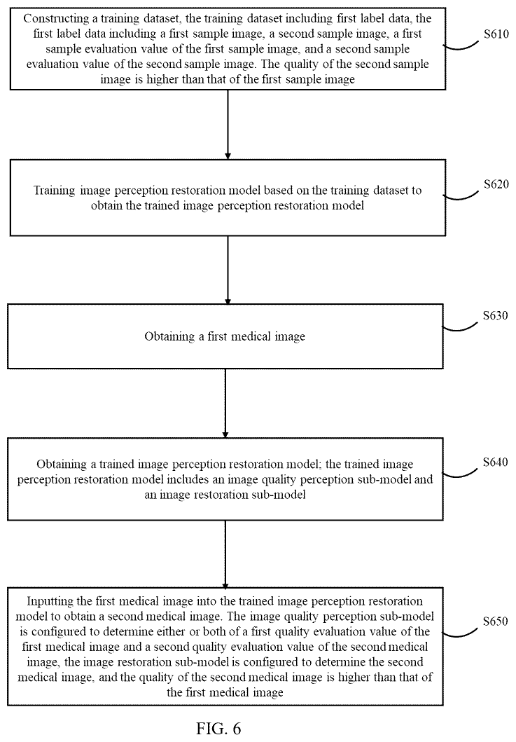

FIG. 6 illustrates a flowchart of another medical image processing method according to an embodiment of the present disclosure. As shown in FIG. 6, the process 600 includes the following step 610 to step 650.

Step 610, constructing a training dataset, the training dataset including first label data, and the first label data including a first sample image, a second sample image, a first sample evaluation value of the first sample image, and a second sample evaluation value of the second sample image. The quality of the second sample image is higher than that of the first sample image.

Step 620, training image perception restoration model based on the training dataset to obtain the trained image perception restoration model.

Step 630, obtaining a first medical image.

Step 640, obtaining a trained image perception restoration model; the trained image perception restoration model includes an image quality perception sub-model and an image restoration sub-model.

Step 650, inputting the first medical image into the trained image perception restoration model to obtain a second medical image. The image quality perception sub-model is configured to determine either or both of a first quality evaluation value of the first medical image and a second quality evaluation value of the second medical image, the image restoration sub-model is configured to determine the second medical image, and the quality of the second medical image is higher than that of the first medical image.

It should be noted that both the image quality perception sub-model and the image restoration sub-model can adopt a structure based on a Convolutional Neural Network (CNN), which is composed of multiple convolutional layers, pooling layers, activation function layers, and de-convolutional layers, for feature extraction and reconstruction of the input image. Additionally, they can be constructed based on a fully connected neural network or other suitable network structures.

In the first label data, the first sample image may be a low-count-rate PET image from a long-axis PET system, and the second sample image may be a high-count-rate PET image from a long-axis PET system; alternatively, the first sample image may be an original medical image with quality issues, and the second sample image may be a high-quality image processed by manual work or a professional algorithm. The evaluation values can be obtained through subjective evaluation by professional doctors or calculation based on a specific image quality assessment algorithm. The quality evaluation of the first sample image is used as the first sample evaluation value of the first sample image, and the quality evaluation of the second sample image is used as the second sample evaluation value of the second sample image. The image perception restoration model is trained based on the first label data to obtain a trained image perception restoration model.

In some embodiments, the training dataset further includes second label data; the second label data includes a third sample image and a third sample evaluation value of the third sample image; training the image perception restoration model based on the training dataset to obtain the trained image perception restoration model includes: training the image perception restoration model based on the first label data to obtain a first image perception restoration model; training the image quality perception sub-model of the first image perception restoration model based on the second label data to obtain the trained image perception restoration model.

In the second label data, the third sample image may be a low-count-rate PET image from a long-axis PET system or an original medical image with quality issues; correspondingly, the third sample evaluation value of the third sample image may be a subjective evaluation of the image by a professional doctor or a value calculated based on a specific image quality assessment algorithm. Alternatively, the third sample image may be a high-count-rate PET image from a long-axis PET system or a high-quality image processed by manual work or a professional algorithm; correspondingly, the third sample evaluation value of the third sample image may be a subjective evaluation of the image by a professional doctor or a value calculated based on a specific image quality assessment algorithm. These sample images can come from different patients, different scanning parts, and different scanning devices to increase data diversity and the generalization ability of the model.

During model training: First, the image perception restoration model is trained based on the first label data to obtain a first image perception restoration model. In the training process, the first sample image is input into the image restoration sub-model of the model, and the model outputs an image with optimized quality. This optimized image is compared with the second sample image, and the difference between them is calculated through a loss function (such as a mean squared error loss function). The model parameters are adjusted through a backpropagation algorithm, so that the restored image output by the model is as close as possible to the second sample image. At the same time, the first sample image and the optimized image are respectively input into the image quality perception sub-model, and the quality evaluation values output by the sub-model are compared with the corresponding actual evaluation values. Similarly, the model parameters are adjusted through the loss function and backpropagation algorithm to make the quality evaluation values as accurate as possible.

Second, the image quality perception sub-model of the first image perception restoration model is trained based on the second label data to obtain the trained image perception restoration model. That is, the network parameters of the image restoration sub-model of the first image perception restoration model are fixed, and the network parameters of the image quality perception sub-model of the first image perception restoration model are fine-tuned based on the second label data. This further optimizes the parameters of the image quality perception sub-model and improves the accuracy of image quality assessment.

In some embodiments, the training dataset further includes third label data; the third label data includes a fourth sample image and a fifth sample image; the quality of the fifth sample image is higher than that of the fourth sample image; training the image perception restoration model based on the training dataset to obtain the trained image perception restoration model includes: training the image perception restoration model based on the first label data to obtain a first image perception restoration model; training the image restoration sub-model of the first image perception restoration model based on the third label data to obtain the trained image perception restoration model.

As mentioned earlier, during model training: First, the image perception restoration model is trained based on the first label data to obtain a first image perception restoration model. Second, the image restoration sub-model of the first image perception restoration model is trained based on the third label data to obtain the trained image perception restoration model. The third label data includes a fourth sample image and a fifth sample image. The fourth sample image may be a low-count-rate PET image from a long-axis PET system, and the fifth sample image may be a high-count-rate PET image from a long-axis PET system; alternatively, the fourth sample image may be an original medical image with quality issues, and the fifth sample image may be a corresponding high-quality image processed by manual work or a professional algorithm. Fixing the network parameters of the image quality perception sub-model of the first image perception restoration model and fine-tuning the network parameters of the image restoration sub-model of the first image perception restoration model based on the third label data can improve the efficiency of model training and make the trained image perception restoration model more accurate.

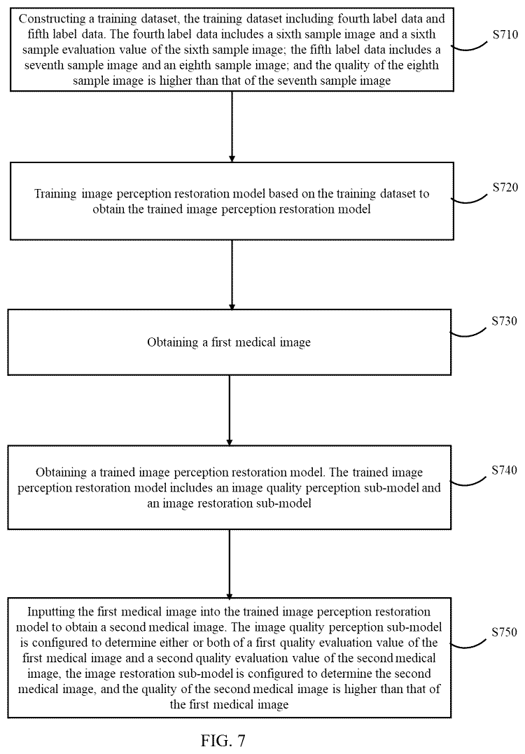

FIG. 7 illustrates a flowchart of another medical image processing method according to an embodiment of the present disclosure. As shown in FIG. 7, the process 700 includes the following step 710 to step 750.

Step 710, constructing a training dataset, the training dataset including fourth label data and fifth label data. The fourth label data includes a sixth sample image and a sixth sample evaluation value of the sixth sample image; the fifth label data includes a seventh sample image and an eighth sample image; and the quality of the eighth sample image is higher than that of the seventh sample image.

Step 720, training image perception restoration model based on the training dataset to obtain the trained image perception restoration model.

Step 730, obtaining a first medical image.

Step 740, obtaining a trained image perception restoration model; the trained image perception restoration model includes an image quality perception sub-model and an image restoration sub-model.

Step 750, inputting the first medical image into the trained image perception restoration model to obtain a second medical image. The image quality perception sub-model is configured to determine either or both of a first quality evaluation value of the first medical image and a second quality evaluation value of the second medical image, the image restoration sub-model is configured to determine the second medical image, and the quality of the second medical image is higher than that of the first medical image.

In some embodiments, training the image perception restoration model based on the training dataset to obtain the trained image perception restoration model includes: training the image quality perception sub-model of the image perception restoration model based on the fourth label data, and training the image restoration sub-model of the image perception restoration model based on the fifth label data to obtain a second image perception restoration model; obtaining the trained image perception restoration model based on the second image perception restoration model.

In some embodiments, the training dataset further includes sixth label data; the sixth label data includes a ninth sample image, a tenth sample image, a ninth sample evaluation value of the ninth sample image, and a tenth sample evaluation value of the tenth sample image; the quality of the tenth sample image is higher than that of the ninth sample image; obtaining the trained image perception restoration model based on the second image perception restoration model includes: training the second image perception restoration model based on the sixth label data to obtain the trained image perception restoration model.

For the model training mentioned earlier, the image perception restoration model is first trained as a whole, and then the network parameters of either the image quality perception sub-model or the image restoration sub-model are fixed, while the network parameters of the other are fine-tuned. Alternatively, one of the image quality perception sub-model and the image restoration sub-model of the image perception restoration model may be trained first, its network parameters fixed, then the other sub-model is trained, and finally the network parameters of the image perception restoration model as a whole are fine-tuned to complete the training of the image perception restoration model.

Specifically, the fourth label data includes a sixth sample image and a sixth sample evaluation value of the sixth sample image. The sixth sample image may be a low-count-rate PET image from a long-axis PET system or an original medical image with quality issues; correspondingly, the sixth sample evaluation value of the sixth sample image may be a subjective evaluation of the image by a professional doctor or a value calculated based on a specific image quality assessment algorithm. Alternatively, the sixth sample image may be a high-count-rate PET image from a long-axis PET system or a high-quality image processed by manual work or a professional algorithm; correspondingly, the sixth sample evaluation value of the sixth sample image may be a subjective evaluation of the image by a professional doctor or a value calculated based on a specific image quality assessment algorithm. The fifth label data includes a seventh sample image and an eighth sample image. The seventh sample image may be a low-count-rate PET image from a long-axis PET system or an original medical image with quality issues; the eighth sample image may be a high-count-rate PET image from a long-axis PET system or a high-quality image processed by manual work or a professional algorithm. The quality of the eighth sample image is higher than that of the seventh sample image.

The image quality perception sub-model of the image perception restoration model is trained based on the fourth label data, and the image restoration sub-model of the image perception restoration model is trained based on the fifth label data, thereby obtaining a second image perception restoration model. It should be noted that in the above model training, the parameters of the image quality perception sub-model of the image perception restoration model may be trained first, these parameters frozen, and then the image restoration sub-model of the image perception restoration model is trained to obtain the second image perception restoration model. At this point, the initial training of the image perception restoration model is completed. The present disclosure does not specifically limit the training order of the sub-models.

To further improve the accuracy of model training, the sixth label data may be configured to train the second image perception restoration model, thereby obtaining the trained image perception restoration model. The sixth label data includes a ninth sample image, a tenth sample image, a ninth sample evaluation value of the ninth sample image, and a tenth sample evaluation value of the tenth sample image. The ninth sample image may be a low-count-rate PET image from a long-axis PET system or an original medical image with quality issues; correspondingly, the ninth sample evaluation value of the ninth sample image may be a subjective evaluation of the image by a professional doctor or a value calculated based on a specific image quality assessment algorithm. The tenth sample image may be a high-count-rate PET image from a long-axis PET system or a high-quality image processed by manual work or a professional algorithm; correspondingly, the tenth sample evaluation value of the tenth sample image may be a subjective evaluation of the image by a professional doctor or a value calculated based on a specific image quality assessment algorithm. The quality of the tenth sample image is higher than that of the ninth sample image. The second image perception restoration model is fine-tuned for some parameters based on the sixth label data to obtain the trained image perception restoration model.

It should be noted that in the present disclosure, there may be data overlap among the various label data. For example, the first sample image or the first sample evaluation value of the first sample image in the first label data may partially overlap with the third sample image or the third sample evaluation value of the third sample image in the second label data. Alternatively, part of the data in the first label data may overlap with part of the data in the sixth label data, and details will not be repeated here.

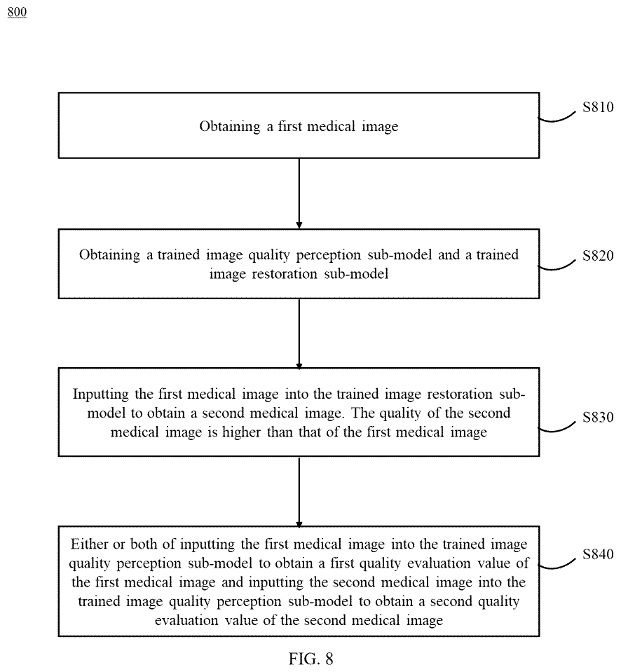

In addition, in combination with the medical image processing method provided in the above embodiments, the embodiments of the present disclosure may also provide a medical image processing system. The system includes: at least one storage medium containing a set of instructions; and at least one processor in communication with the at least one storage medium. When executing the set of instructions, the at least one processor is configured to cause the system to perform the following operations. FIG. 8 illustrates a flowchart of another medical image processing method according to an embodiment of the present disclosure. As shown in FIG. 8, the process 800 includes the following step 810 to step 840.

Step 810, obtaining a first medical image.

Step 820, obtaining a trained image quality perception sub-model and a trained image restoration sub-model.

Step 830, inputting the first medical image into the trained image restoration sub-model to obtain a second medical image. The quality of the second medical image is higher than that of the first medical image.

Step 840, either or both of inputting the first medical image into the trained image quality perception sub-model to obtain a first quality evaluation value of the first medical image and inputting the second medical image into the trained image quality perception sub-model to obtain a second quality evaluation value of the second medical image.

In this embodiment, image quality evaluation and image quality optimization can be performed through the trained image quality perception sub-model and the trained image restoration sub-model respectively. The specific model training dataset and training process are similar to those described earlier, and details will not be repeated here.

In some embodiments, the trained image quality perception sub-model includes a third image quality perception sub-model and a fourth image quality perception sub-model. The first quality evaluation value of the first medical image is determined by the third image quality perception sub-model, and the second quality evaluation value of the second medical image is determined by the fourth image quality perception sub-model.

In some embodiments, the network parameters of the third image quality perception sub-model and the fourth image quality perception sub-model in the trained image quality perception sub-model are the same.

The trained image quality perception sub-model may include different image quality perception sub-models, such as a third image quality perception sub-model and a fourth image quality perception sub-model. In this case, the first quality evaluation value of the first medical image and the second quality evaluation value of the second medical image may be determined by different image quality perception sub-models respectively. The network parameters of the third image quality perception sub-model and the fourth image quality perception sub-model in the trained image quality perception sub-model may be the same. This structural design can enhance the model's ability to learn image features and improve the accuracy of image quality assessment. Additionally, in other embodiments, the third image quality perception sub-model and the fourth image quality perception sub-model may have different network parameters.

In some embodiments, the trained image perception restoration model is obtained based on the trained image quality perception sub-model and the trained image restoration sub-model.

In the above embodiments, the trained image quality perception sub-model and the trained image restoration sub-model may be integrated to obtain the trained image perception restoration model. The first medical image is input into the trained image perception restoration model to obtain a second medical image with optimized quality, as well as the first quality evaluation value of the first medical image and/or the second quality evaluation value of the second medical image.

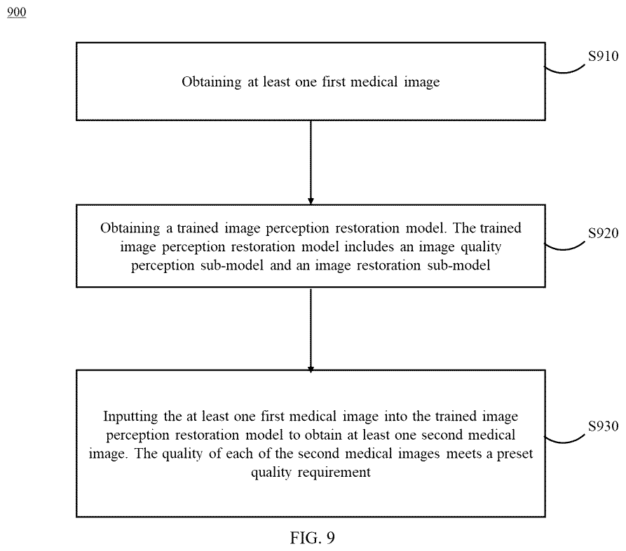

The present disclosure also provides a medical image processing method. FIG. 9 illustrates a flowchart of another medical image processing method according to an embodiment of the present disclosure. As shown in FIG. 9, the process 900 includes the following step 910 to step 930.

Step 910, obtaining at least one first medical image.

The first medical image may be any type of medical image, such as a CT image, an MRI image, or an ultrasound image.

Step 920, obtaining a trained image perception restoration model; the trained image perception restoration model includes an image quality perception sub-model and an image restoration sub-model.

In this step, the training dataset and training process of the model are similar to those described in the previous embodiments. For example, a training set containing the first label data and the second label data is used, and through multiple rounds of training and parameter adjustment, a well-performing trained image perception restoration model is obtained.

Step 930, inputting the at least one first medical image into the trained image perception restoration model to obtain at least one second medical image. The quality of each of the second medical images meets a preset quality requirement.

In this step, the at least one first medical image is input into the trained image perception restoration model. After feature extraction, feature processing, and image reconstruction processes similar to those described earlier, a second medical image with optimized quality is obtained, along with the first quality evaluation value of the first medical image and/or the second quality evaluation value of the second medical image. Specifically, when the first medical image is a PET image, the image restoration sub-model in the trained image perception restoration model addresses issues such as noise and blurriness in the PET image to improve image quality. The first medical image and/or the second medical image are respectively input into the image quality perception sub-model, and through steps such as feature extraction and feature processing, the first quality evaluation value of the first medical image and/or the second quality evaluation value of the second medical image are obtained. By comparing these two evaluation values, the effect of the image restoration sub-model on processing PET scan images can be evaluated, and at the same time, quantitative information about image quality is provided to doctors, helping them better judge whether the image is suitable for diagnosis.

In particular, the image quality of each of the multiple second medical images output by the trained image perception restoration model meets the preset quality requirement. For example, the resolution, clarity, signal-to-noise ratio, etc., of each second medical image are better than the corresponding threshold requirements.

In some embodiments, the at least one first medical image is a plurality of first medical images with different qualities; the plurality of first medical images with different qualities are input into the trained image perception restoration model to obtain a plurality of second medical images, and the plurality of second medical images meet the same quality requirement.

In this embodiment, the plurality of first medical images with different qualities may be medical images with varying quality acquired by different devices, or medical images with different count rates acquired by the same type of device. By using a uniformly trained image perception restoration model to optimize the image quality of the plurality of first medical images with different qualities, and evaluating the optimized medical images using a unified quality evaluation standard, the problem in the prior art of inconsistent image quality output by devices due to different devices or users is solved, and unified quality optimization and quality evaluation of medical images are realized. In one possible scenario, the plurality of first medical images with different qualities are medical images acquired by different short-axis PET devices. The optimized PET images can reach or approach the quality corresponding to images acquired using long-axis PET scanning devices. Based on this solution, users can obtain images with quality close to that of long-axis PET devices through short-axis PET devices, significantly reducing user costs.

In some embodiments, the image quality perception sub-model includes a first image quality perception sub-model and a second image quality perception sub-model. The first quality evaluation value of the first medical image is determined by the first image quality perception sub-model, and the second quality evaluation value of the second medical image is determined by the second image quality perception sub-model.

In some embodiments, the network parameters of the first image quality perception sub-model and the second image quality perception sub-model in the trained image perception restoration model are the same.

The image quality perception sub-model may include different image quality perception sub-models, such as a first image quality perception sub-model and a second image quality perception sub-model. In this case, the first quality evaluation value of the first medical image and the second quality evaluation value of the second medical image may be determined by different image quality perception sub-models respectively. The network parameters of the first image quality perception sub-model and the second image quality perception sub-model in the trained image perception restoration model may be the same. This structural design can enhance the model's ability to learn image features and improve the accuracy of image quality assessment. Additionally, in other embodiments, the first image quality perception sub-model and the second image quality perception sub-model may have different network parameters. In still other embodiments, the first image quality perception sub-model and the second image quality perception sub-model may adopt the same or different network structures.

In some embodiments, inputting the at least one first medical image into the trained image perception restoration model to obtain the at least one second medical image and the second quality evaluation value of the second medical image includes: after determining the second medical image based on the image restoration sub-model, inputting the second medical image into the image quality perception sub-model to obtain the second quality evaluation value of the second medical image.

When the image quality perception sub-model includes only one model (i.e., a single image quality perception sub-model is required to determine both the first quality evaluation value of the first medical image and the second quality evaluation value of the second medical image), after the at least one first medical image is input into the trained image perception restoration model, the first quality evaluation value of the first medical image may first be determined based on the image quality perception sub-model; at the same time, the second medical image with optimized quality is determined based on the image restoration sub-model; subsequently, the second medical image is input into the image quality perception sub-model to obtain the second quality evaluation value of the second medical image. In addition, the second medical image with optimized quality may first be determined based on the image restoration sub-model; then both the first medical image and the second medical image are input into the image quality perception sub-model to obtain the first quality evaluation value of the first medical image and the second quality evaluation value of the second medical image. Alternatively, only the second medical image may be input into the image quality perception sub-model to obtain the second quality evaluation value of the second medical image, without evaluating the quality of the first medical image.

The technical solution of the present disclosure will be further described below by taking the image perception restoration model as a convolutional neural network model and the medical device as a PET device as an example.

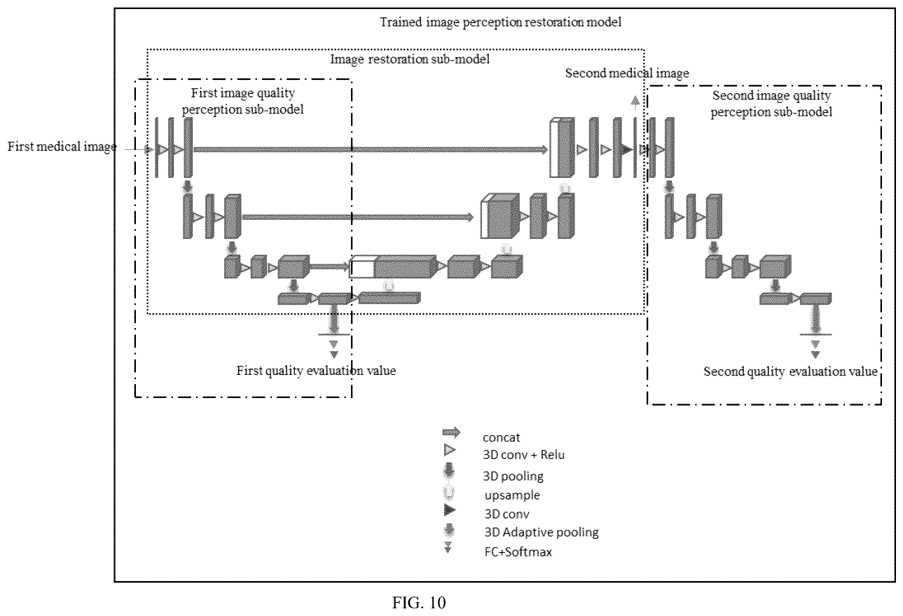

The structure of the image perception restoration model is shown in FIG. 10. The image perception restoration model is a network structure that includes both an image quality perception sub-model and an image restoration sub-model. It can evaluate the image quality of the input image, optimize the image quality based on the evaluation, and output the optimized image and the corresponding image quality score. The network structure in FIG. 10 includes an image restoration sub-model and two image quality perception sub-models (a first image quality perception sub-model and a second image quality perception sub-model). The network structures of the first image quality perception sub-model and the second image quality perception sub-model may be the same; further, the first image quality perception sub-model and the second image quality perception sub-model may share weight parameters, i.e., their network parameters may be the same. The blue bars in FIG. 10 can be understood as feature maps obtained after corresponding feature processing, and the concatenation of white bars and blue bars can be understood as feature combination.

The specific training process of the image perception restoration model is as follows: Based on long-term collected data from a long-axis PET system, random sampling at different sampling rates is performed, and offline image reconstruction is conducted to obtain a series of long-axis PET images under different counts. At the same time, reconstructed images from different short-axis systems (under acquisition conditions commonly used in actual hospitals) are also prepared. Thus, a database containing images of varying quality from different systems is established, and part of the data in this database consists of paired data of the same individual under different counting conditions. From the perspective of doctors and/or technicians, an image quality correlation table is established. The purpose of this table is to correlate long-axis PET images under different counts with different scores, where a lower score indicates poorer image quality. The data in the database can be represented as fully labeled data (x, x_score, y, y_score) and semi-labeled data (x, x_score); in addition, the semi-labeled data can also be (x, y). The purpose of semi-labeled data is to enrich the dataset and improve its accuracy. Here, x is the image data in the database (i.e., the first medical image), x_score is the first evaluation value (i.e., the quality evaluation value of the first medical image), which is the score given by doctors and/or technicians for image x; y is the restoration target of image x (i.e., the second medical image) (e.g., x is a low-count long-axis image with low quality, and y is a higher-count image of the same individual with better quality); y_score is the second evaluation value (i.e., the quality evaluation value of the second medical image). It should be understood that the fully labeled data corresponds to the first label data described earlier, and the semi-labeled data corresponds to the second label data or third label data described earlier.

Based on the fully labeled data and semi-labeled data generated above, the training of the image perception restoration model is conducted as follows: First, the complete image perception restoration model is trained based on (x, x_score, y, y_score). Then, the network parameters of either the image quality perception sub-model or the image restoration sub-model in the model are fixed, and the network parameters of the other are fine-tuned. In addition, one of the image quality perception sub-model and the image restoration sub-model may be trained first, its network parameters fixed, then the other sub-model is trained, and finally the network parameters of the image perception restoration model as a whole are fine-tuned to complete the training of the image perception restoration model. For example, based on the fully labeled and semi-labeled data, the image perception restoration model is trained in stages: First, only the semi-labeled data (x, x_score) is configured to train the network parameters of the quality perception sub-model, which are then fixed. Next, the semi-labeled data (x, y) is configured to train the network parameters of the image restoration sub-model. At this point, the initial training of the entire image perception restoration model is completed. Subsequently, the fully labeled data (x, x_score, y, y_score) is configured to fine-tune the entire image perception restoration model to obtain the final image perception restoration model.

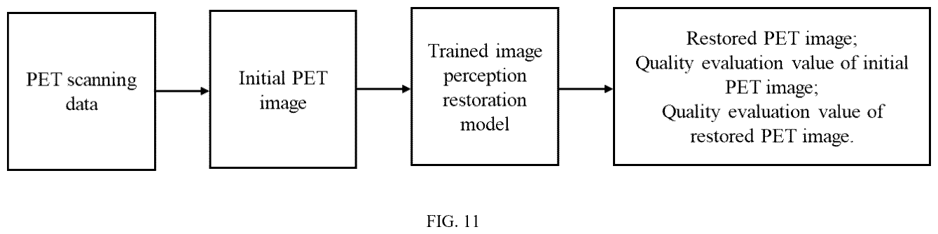

The way the trained image perception restoration model is used in the workflow can be as shown in FIG. 11. FIG. 11 is a schematic diagram of an application according to an embodiment of the present disclosure. The trained image quality perception restoration model can be cascaded after PET reconstruction and image output. After PET scanning and reconstruction are completed, the PET image is input into the trained image perception restoration model, and the end user will finally obtain a PET image with improved quality, along with a quality assessment of the corresponding PET image.

For equipment manufacturers, they ideally want to provide users with high-quality images. Currently, with the launch of long-axis PET systems (high-end equipment), PET images that are closest to the Ground Truth (real data) can be obtained. However, most users still use mid-to-low-end equipment. Based on this solution, the image quality of long-axis PET can be transferred to different short-axis systems, enabling simultaneous consideration of image quality perception and image restoration. The solution can automatically perceive different image qualities (even images from devices of different manufacturers) and perform automatically matched image restoration, so that the image quality is restored to a unified level.

The embodiments of the present disclosure also provide a medical image processing device, which is configured to implement the above embodiments and preferred implementations. Descriptions that have already been provided will not be repeated. The terms “module”, “unit”, “sub-unit”, etc., used below may be a combination of software and/or hardware that implements predetermined functions. Although the device described in the following embodiments is preferably implemented in software, implementation in hardware or a combination of software and hardware is also possible and contemplated.

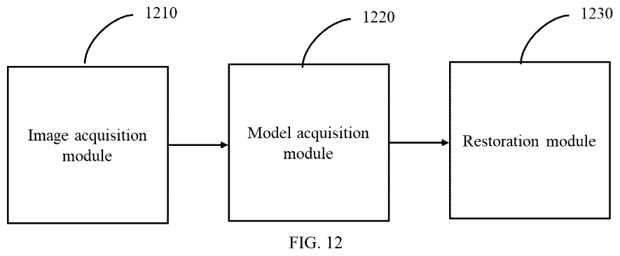

FIG. 12 is a structural block diagram of a medical image processing device according to an embodiment of the present disclosure. As shown in FIG. 12, the device includes an image acquisition module 1210, a model acquisition module 1220, and a restoration module 1230.

The image acquisition module 1210 is configured to obtain a first medical image.

The model acquisition module 1220 is configured to obtain a trained image perception restoration model, and the trained image perception restoration model includes an image quality perception sub-model and an image restoration sub-model.

The restoration module 1230 is configured to input the first medical image into the trained image perception restoration model to obtain a second medical image; where the image quality perception sub-model is configured to determine either or both of a first quality evaluation value of the first medical image and a second quality evaluation value of the second medical image, the image restoration sub-model is configured to determine the second medical image, and the quality of the second medical image is higher than that of the first medical image.

It should be noted that the above modules may be functional modules or program modules, and can be implemented by software or hardware. For modules implemented by hardware, the above modules may be located in the same processor; alternatively, the above modules may be located in different processors in any combination. For specific examples in the embodiments of the present disclosure, reference may be made to the examples described in the above embodiments and optional implementations, and details will not be repeated in the embodiments of the present disclosure.

In addition, in combination with the medical image processing method provided in the above embodiments, the embodiments of the present disclosure may also provide a storage medium for implementation. A computer program is stored on the storage medium; when executed by a processor, the computer program implements any one of the medical image processing methods in the above embodiments.

It should be understood that the specific embodiments described herein are only used to explain the present disclosure and are not intended to limit it. Based on the embodiments provided in the present disclosure, all other embodiments obtained by those of ordinary skill in the art without creative work shall fall within the protection scope of the present disclosure.

Obviously, the accompanying drawings are only some examples or embodiments of the present disclosure. For those of ordinary skill in the art, the present disclosure can also be applied to other similar scenarios based on these drawings without creative work. In addition, it can be understood that although the work involved in the development process may be complex and lengthy, for those of ordinary skill in the art, some design, manufacturing, or production modifications made based on the technical content disclosed in the present disclosure are only conventional technical means and should not be considered as insufficient disclosure of the content of the present disclosure.

The term “embodiment” in the present disclosure means that a specific feature, structure, or characteristic described in connection with the embodiment can be included in at least one embodiment of the present disclosure. The appearance of this phrase in various positions in the specification does not necessarily refer to the same embodiment, nor does it refer to an embodiment that is mutually exclusive or independent of other embodiments. Those of ordinary skill in the art can clearly or implicitly understand that the embodiments described in the present disclosure can be combined with other embodiments without conflict.

The above-described embodiments only represent several implementation manners of the present disclosure, and their descriptions are relatively specific and detailed, but they should not be construed as limiting the scope of patent protection. It should be noted that for those of ordinary skill in the art, several modifications and improvements can be made without departing from the concept of the present disclosure, and these all fall within the protection scope of the present disclosure. Therefore, the protection scope of the present disclosure should be subject to the appended claims.

It should be noted that the processes 200, 600, 700, 800 and 900 and the descriptions thereof are provided for the purposes of illustration, and not intended to limit the scope of the present disclosure. For persons having ordinary skills in the art, various modifications and changes in the forms and details of the application of the above method and system may occur without departing from the principles of the present disclosure. However, those variations and modifications also fall within the scope of the present disclosure. For example, the operations of the illustrated processes are intended to be illustrative. In some embodiments, the processes may be accomplished with one or more additional operations not described, and/or without one or more of the operations discussed. Additionally, the order in which the operations of the processes and regarding descriptions are not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment”, “an embodiment”, and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module”, “unit”, “component”, “device”, or “system”. Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an subject oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

In some embodiments, the numbers expressing quantities or properties used to describe and claim certain embodiments of the application are to be understood as being modified in some instances by the term “about”, “approximate”, or “substantially”. For example, “about”, “approximate”, or “substantially” may indicate a certain variation (e.g., ±1%, ±5%, ±10%, or +20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value”.

What is claimed is:

1. A method for medical image processing, implemented on a machine comprising one or more processors and one or more storage devices, the method comprising:
obtaining a first medical image;
obtaining a trained image perception restoration model; the trained image perception restoration model comprises an image quality perception sub-model and an image restoration sub-model; and
inputting the first medical image into the trained image perception restoration model to obtain a second medical image; wherein the image quality perception sub-model is configured to determine either or both of a first quality evaluation value of the first medical image and a second quality evaluation value of the second medical image, the image restoration sub-model is configured to determine the second medical image, and the quality of the second medical image is higher than that of the first medical image.

2. The method of claim 1, wherein the first quality evaluation value is configured to evaluate the quality of the first medical image, the second quality evaluation value is configured to evaluate the quality of the second medical image, and the second quality evaluation value is higher than the first quality evaluation value.

3. The method of claim 1, wherein the image quality perception sub-model comprises a first image quality perception sub-model and a second image quality perception sub-model, the first quality evaluation value of the first medical image is determined by the first image quality perception sub-model, and the second quality evaluation value of the second medical image is determined by the second image quality perception sub-model.

4. The method of claim 3, wherein network parameters of the first image quality perception sub-model and the second image quality perception sub-model in the trained image perception restoration model are the same.

5. The method of claim 1, wherein the image quality perception sub-model is configured to determine the second quality evaluation value of the second medical image comprises:
determining the second medical image by the image restoration sub-model; and
inputting the second medical image into the image quality perception sub-model to obtain the second quality evaluation value of the second medical image.

6. The method of claim 1, further comprising:
constructing a training dataset, the training dataset comprising first label data, the first label data comprising a first sample image, a second sample image, a first sample evaluation value of the first sample image, and a second sample evaluation value of the second sample image; wherein the quality of the second sample image is higher than that of the first sample image; and
training image perception restoration model based on the training dataset to obtain the trained image perception restoration model.

7. The method of claim 6, wherein the training dataset further comprises second label data; the second label data comprises a third sample image and a third sample evaluation value of the third sample image; and
the training image perception restoration model according to the training dataset to obtain the trained image perception restoration model further comprises:
training image perception restoration model according to the first label data to obtain a first image perception restoration model; and
training the image quality perception sub-model of the first image perception restoration model according to the second label data to obtain the trained image perception restoration model.

8. The method of claim 6, wherein the training dataset further comprises third label data; the third label data comprises a fourth sample image and a fifth sample image; the quality of the fifth sample image is higher than that of the fourth sample image; and
the training image perception restoration model according to the training dataset to obtain the trained image perception restoration model further comprises:
training image perception restoration model according to the first label data to obtain a first image perception restoration model; and
training the image restoration sub-model of the first image perception restoration model according to the third label data to obtain the trained image perception restoration model.

9. The method of claim 1, further comprising:
constructing a training dataset, the training dataset comprising fourth label data and fifth label data; wherein the fourth label data comprises a sixth sample image and a sixth sample evaluation value of the sixth sample image; the fifth label data comprises a seventh sample image and an eighth sample image; the quality of the eighth sample image is higher than that of the seventh sample image; and
training image perception restoration model based on the training dataset to obtain the trained image perception restoration model.

10. The method of claim 9, wherein the training image perception restoration model according to the training dataset to obtain the trained image perception restoration model further comprises:
training the image quality perception sub-model and the image restoration sub-model of the image perception restoration model to obtain a second image perception restoration model; wherein the image quality perception sub-model is trained according to the fourth label data and the image restoration sub-model is trained according to the fifth label data; and
obtaining the trained image perception restoration model based on the second image perception restoration model.

11. The method of claim 10, wherein the training dataset further comprises sixth label data; the sixth label data comprises a ninth sample image, a tenth sample image, a ninth sample evaluation value of the ninth sample image, and a tenth sample evaluation value of the tenth sample image; wherein the quality of the tenth sample image is higher than that of the ninth sample image; and
the obtaining the trained image perception restoration model based on the second image perception restoration model comprises:
training the second image perception restoration model according to the sixth label data to obtain the trained image perception restoration model.

12. A system for medical image processing, comprising:
at least one storage devices comprising a set of instructions; and
at least one processor in communication with the at least one storage devices, wherein, when executing the set of instructions, the at least one processor is configured to cause the system to perform the following operations:
obtaining a first medical image;
obtaining a trained image quality perception sub-model and a trained image restoration sub-model;
inputting the first medical image into the trained image restoration sub-model to obtain a second medical image; wherein the quality of the second medical image is higher than that of the first medical image; and
either or both of inputting the first medical image into the trained image quality perception sub-model to obtain a first quality evaluation value of the first medical image and inputting the second medical image into the trained image quality perception sub-model to obtain a second quality evaluation value of the second medical image.

13. The system of claim 12, wherein the trained image quality perception sub-model comprises a third image quality perception sub-model and a fourth image quality perception sub-model, the first quality evaluation value of the first medical image is determined by the third image quality perception sub-model, and the second quality evaluation value of the second medical image is determined by the fourth image quality perception sub-model.

14. The system of claim 13, wherein network parameters of the third image quality perception sub-model and the fourth image quality perception sub-model in the trained image quality perception sub-model are the same.

15. The system of claim 12, further comprising: obtaining a trained image perception restoration model based on the trained image quality perception sub-model and the trained image restoration sub-model.

16. A method for medical image processing, comprising:
obtaining at least one first medical image;
obtaining a trained image perception restoration model; the trained image perception restoration model comprises an image quality perception sub-model and an image restoration sub-model; and
inputting the at least one first medical image into the trained image perception restoration model to obtain at least one second medical image; wherein the quality of each of the second medical images meets a preset quality requirement.

17. The method of claim 16, wherein the at least one first medical image of a plurality of first medical images with different qualities; and the method further comprises:
inputting the plurality of first medical images with different qualities into the trained image perception restoration model to obtain a plurality of second medical images, and the plurality of second medical images meet the same quality requirement.

18. The method of claim 16, wherein the image quality perception sub-model comprises a first image quality perception sub-model and a second image quality perception sub-model, a first quality evaluation value of the first medical image is determined by the first image quality perception sub-model, and a second quality evaluation value of the second medical image is determined by the second image quality perception sub-model.

19. The method of claim 18, wherein network parameters of the first image quality perception sub-model and the second image quality perception sub-model in the trained image perception restoration model are the same.

20. The method of claim 18, further comprising:
inputting the at least one second medical image into the image quality perception sub-model to obtain the second quality evaluation value of the second medical image.
